(19) United States
(12) Plant Patent Application Publication
(10) Pub. No.: US 2002/0188995 P1
(43) Pub. Date: Dec. 12, 2002

(54) Petunia plant named 'Sunlapur'

(76) Inventor: Kiyoshi Miyazaki, Hikone-shi (JP)

Correspondence Address: BURNS, DOANE, SWECKER & MATHIS, L.L.P., P.O. Box 1404, Alexandria, VA, 22313-1404, US

(21) Appl. No.: 09/878,237

(22) Filed: Jun. 12, 2001

(51) Int. Cl. A01H 5/00

(52) U.S. Cl. PLT/356

(57) ABSTRACT

‘SUNLAPUR’ is a decumbent plant, with very large and vivid purplish red flowers, which is suitable for potting and flower bedding. The spreading area of the plant is very large. This plant has abundant branching. The length of internodes is 2.8cm. The leaf shape is lanceolate with some pubescence on the surface, having small petiole. Leaf color is dark green.

The flowers are single and very large. Facing direction of flowers is slightly upward to horizontal. Ground color of flower is vivid reddish purple with dark purple vein. The bottom color of inside of throat is strong purple and the outside color of corolla tube is reddish purple. Shape of petal tip is acute, lobation is deep, and waving is medium.

The plant exhibits early flowering. It has a high resistance to rain, heat, disease and moderate resistance to pests.

### BACKGROUND OF THE VARIETY

The present invention relates to a new and distinct variety of petunia plant obtained from crossing a wild-type petunia plant native to Brazil, as the male parent, and a cultivar ‘Titan Red’ as the female parent.

The petunia is a very popular plant and is used for flower bedding and potting in the summer season. There are only a few varieties of the petunia plant which do not have an upright growth habit and which have a high resistance to rain, heat, cold, and diseases. The petunia previously filed, i.e., the ‘Revolution’ series [(Revolution Purplepink (US Pat. No, Plant 6,915), Revolution Brilliantpink (US Pat. No, Plant 6,914), Revolution Brilliantpink-mini (US Pat. No, Plant 6,899)) is a decumbent type plant having long stems, a lower plant height, abundant branching, and a high resistance to heat, cold and rain. However, there are only a few varieties having a great profusion of flowers, vivid reddish purple color and large flower and a high resistance to rain, heat, cold and diseases. Accordingly, this invention was aimed at obtaining a new variety having a vivid reddish purple color and large flower, together with the above features.

The new variety of petunia plant according to this invention originated from crossing a wild-type petunia plant, native to Brazil and which had decumbent habit, as the male parent and a cultivar ‘Titan Red’, produced from a seedling, as the female parent in April 1995. From that crossing, 384 seedlings were obtained in July 1995. A trial by flower potting was carried out and 15 seedlings were selected. These 15 seedlings were propagated by cutting, and grown, as a trial, by flower bedding and in a container. The botanical characteristics of these plants were examined, using similar variety ‘Revolution brilliantpink’ and ‘Revolution brilliantpink- mini’ for comparison. Finally, only one petunia plant was selected in December 1996. As a result, it was concluded that this new variety is distinguishable from other varieties especially in flower size, and this variety of petunia plant was named ‘SUNLAPUR’.

The wild-type petunia plant, native to Brazil and used as male parent is presently maintained at Oumi Nursery Center of SUNTORY Ltd. residing at 863-1, Aza-Iketani, Oomori-cho, Yokaichi-shi, Shiga-ken, Japan.

This new variety of petunia plant named ‘SUNLAPUR’ was asexually reproduced by cutting at the aforementioned Oumi Nursery Center of SUNTORY Ltd. and the homogeneity and stability thereof were confirmed.

In the following description, the color-coding is in accordance with the Horticultural Color Chart of The Royal Horticultural Society, London, England (R.H.S. Color Chart), and the Inter-Society Color Council-Nation Bureau of Standards Color Name (I.S.C.C.-N.B.S. Color Name). A color chart based on The Japan Color Standard for Horticultural Plants (J.H.S. Color Chart) is also added for reference.

### SUMMARY OF THE VARIETY

Disclosed herein is a decumbent type petunia plant named ‘ISUNLAPUR’ having very large vivid reddish purple flowers. The plant has abundant branching, a great profusion of blooms, and the whole bush remains in bloom for a considerable period of time. The plant is highly resistant to rain, heat, cold and disease. This new variety of petunia plant is suitable for potting, hanging pots and flower bedding.

### BRIEF DESCRIPTION OF THE DRAWINGS

FIG. 1 is photograph giving a partial view of the new variety of petunia plant named ‘SUNLAPUR’;

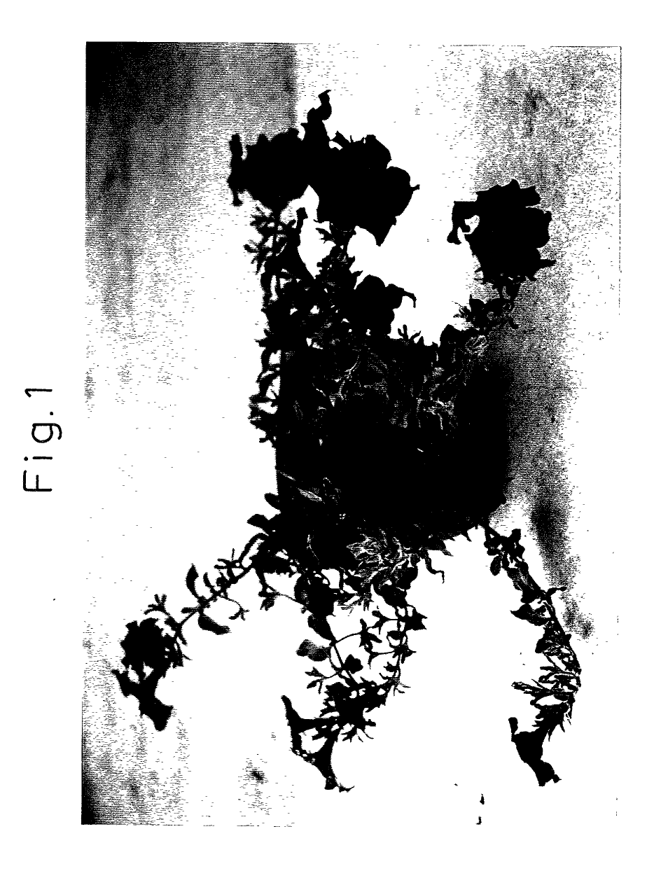

FIG. 2 is a photograph of flowers of the new variety of petunia plant named ‘SUNLAPUR’.

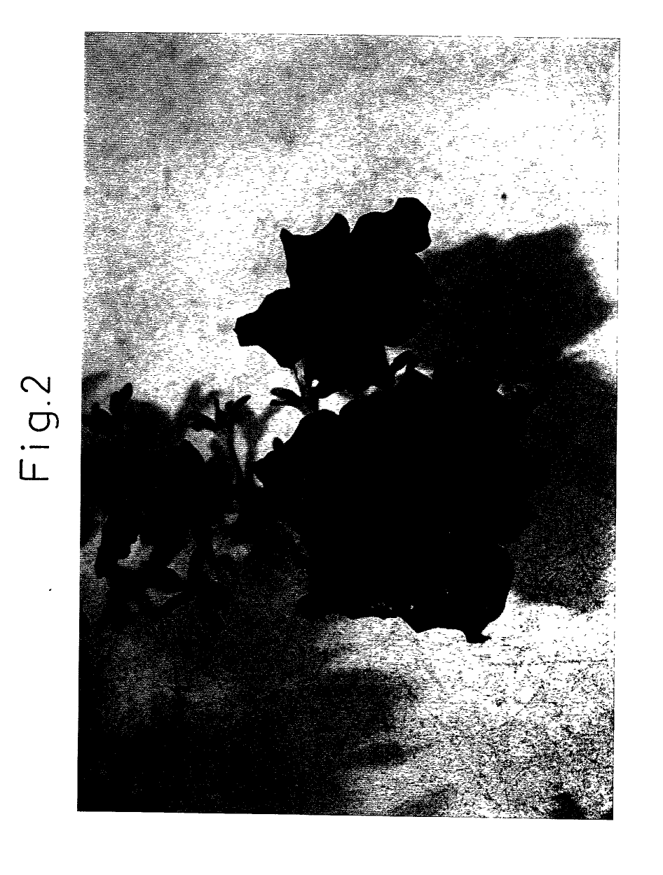

### DESCRIPTION OF THE VARIETY

The new variety of Petunia plant ‘SUNLAPUR’ is different from a similar variety, ‘Revolution Brilliantpink’ in the following points,

1. The length of main stem from the base to the first flowering node of ‘SUNLAPUR’ is 14 cm and is shorter than the 50 cm of ‘Revolution Brilliant pink’.

2. There is no anthocyanin coloration on the stem of ‘SUNLAPUR’ as against the stem of ‘Revolution Brilliant pink’ that shows some anthocyanin coloration.

3. The shape of leaves of ‘SUNLAPUR’ is lanceolate as against the leaf of ‘Revolution Brilliant pink’ is elliptic.

4. The attaching angle of the upper leaf of the main stem of ‘SUNLAPUR’ is horizontal to droopy as against the leaf of ‘Revolution Brilliantpink’ that is attached slightly upward to horizontal.

5. The surface color of leaf of ‘SUNLAPUR’ is dark green (RHS C.C. No.137A) as against the color of ‘Revolution Brilliantpink’ which is dark yellowish green (RHS C.C. No.146A).

6. The flower size of ‘SUNLAPUR’ is larger than that of ‘Revolution Brilliantpink’.

7. The flower color of ‘SUNLAPUR’ is vivid reddish purple (RHS C.C. No.74A) with dark purple vein (RHS C.C. No.79B) as against the flower color of ‘Revolution Brilliantpink’ that shows vivid reddish purple (RHS C.C. No.74A) without patterns.

8. The bottom color of the inside of the throat of ‘SUNLAPUR’ is strong purple (RHS C.C. No.83D) as against the throat of ‘Revolution Brilliant pink’ that shows dark grayish purple (RHS C.C. No.79A).

9. The outside color of the corolla tube of ‘SUNLAPUR’ is reddish purple (RHS C.C. No.77A) as against the color of the corolla tube of ‘Revolution Brilliantpink’ that shows deep purplish pink (RHS C.C. No.70C).

10. The lobation of the petals of ‘SUNLAPUR’ is deeper than that of ‘Revolution Brilliantpink’.

The new variety of Petunia plant ‘SUNLAPUR’ is different from a similar variety, ‘Revolution Brilliantpink mini’ in following points,

1. The pubesence of the leaf of ‘SUNLAPUR’ is thicker than that of ‘Revolution Brilliantpink mini’.

2. The flower size of ‘SUNLAPUR’ is larger than that of ‘Revolution Brilliantpink mini’.

3. The flower color of ‘SUNLAPUR’ is vivid reddish purple (RHS C.C. No.74A) with dark purple vain (RHS C.C. No.79B) as against the flower of ‘Revolution Brilliantpink-mini’ that shows strong reddish purple (RHS C.C. No.72A) with dark grayish purple vein (RHS C.C. No.79A).

4. The bottom color of the inside of the throat of ‘SUNLAPUR’ is strong purple (RHS C.C. No.83D) as against the throat of ‘Revolution Brilliantpink-mini’ that shows moderate purple (RHS C.C. No.83B).

5. The outside color of corolla tube of ‘SUNLAPUR’ is reddish purple (RHS C.C. No.77A) as against the corolla tube of ‘Revolution Brilliantpink-mini’ that shows strong reddish purple (RHS C.C. No.77B).

6. The shape of petal tip of ‘SUNLAPUR’ is acute as against the petal tip of ‘Revolution Brilliantpink mini’ is obverse.

7. The lobation of the petals of ‘SUNLAPUR’ is deeper than that of ‘Revolution Brilliantpink- mini’.

8. The pistil and stamen of ‘SUNLAPUR’ are thinner than that of ‘Revolution Brilliantpink -mini’. ‘SUNLAPUR’ is a decumbent plant, with very large and vivid redish flowers, which is suitable for potting and flower bedding. The spreading area of the plant is very large and the plant height is 11.5cm at the blooming time of the 5^{th} to the 10^{th} flower. Diameter of stem is 2.8 mm, with no anthocyanin coloration. This plant has abundant branching. The length of the internode is 2.8 cm.

The leaves are medium size, 66mm in length, 27 mm in width, 0.4 mm in thickness. Leaf shape is lanceolate, with some pubescence on the surface, and has a small petiole. Leaf color is dark green (RHS C.C. No.137A).

The flowers are single and very large, 10 cm in diameter. The facing direction of flowers is slightly upward to horizontal. The ground color of flower is vivid reddish purple (RHS C.C. No.74A) with dark purple (RHS C.C. No.79B) vein. The bottom color of inside of throat is strong purple (RHS C.C. No.83D) and the outside color of corolla tube is reddish purple (RHS C.C. No.77A). Shape of petal tip is acute, lobation is deep, and waving is medium. Shape of calyx is medium. Shape of stamen is normal and number of stamens are 5. Shape of pistil is normal and number of pistil is one. Relative position of stigma and anthers is concave. Length of peduncle is 21 mm, 1.6 mm in diameter.

The plant exhibits early flowering. It has a high resistance to rain, heat, disease and moderate resistance to pests.

1. The new and distinct variety of Petunia plant, substantially as herein illustrated and described, characterized particularly as to novelty by (A) being decumbent, (B) having an abundant branching and a great profusion of blooms, the whole bush remaining in bloom for considerable period of time, (C) having flowers that are single and very large, the petals having a vivid reddish purple color with dark purple vein, and (D) having high resistance to rain, heat, disease.
